(19) United States
(12) Patent Application Publication
(10) Pub. No.: US 2008/0040839 A1
(43) Pub. Date: Feb. 21, 2008

(54) FLEXIBLE COOLING GARMENT

(76) Inventor: Frank Gordon, Delmar, CA (US)

Correspondence Address: ERIC HANSCOM, 7395 PORTAGE WAY, CARLSBAD, CA, 92011, US

(21) Appl. No.: 11/766,103

(22) Filed: Jun. 21, 2007

Related U.S. Application Data
(60) Provisional application No. 60/837,730, filed on Aug. 16, 2006.

(51) Int. Cl. A41D 13/00; A41D 1/00; A61F 7/00

(52) U.S. Cl. 2/455; 2/69; 607/108

(57) ABSTRACT

The embodiments of the invention provide a wearable garment to evaporatively cool humans and animals. The garment contains a body portion and an attachment portion. The body portion is generally defined by at least one outer seam. The body portion contains three layers—an outer layer, a middle layer, and an inner layer. The outer layer is comprised of a breathable fabric. The middle layer is comprised of a substantially hydrophilic material. The inner layer is substantially hydrophobic, preferably comprising a layer of finely woven substantially hydrophobic fibrous material. The attachment portion contains at least one means for attaching the garment to a wearer. To activate the cooling process, the garment is first immersed in water, wherein the middle layer absorbs water. The wearer then wrings the excess water from the garment and places the garment on the desired body region to be cooled.

### CROSS REFERENCE TO RELATED APPLICATIONS

This patent claims priority to U.S. Provisional Patent Application No. 60/837,730, filed on Aug. 16, 2007, which is incorporated by reference into this Utility Patent Application.

### STATEMENT REGARDING FEDERALLY SPONSORED RESEARCH OR DEVELOPMENT

This invention was not federally sponsored.

### BACKGROUND OF INVENTION

1. Field of the Invention

The embodiments of the invention relate to the field of garments, more particularly, garments for use by humans or animals. More specifically, the embodiments of the present invention relate to flexible garments that can be used to cool humans and animals.

2. Description of the Related Art

Humans often complain about being too hot or too cold. While it is relatively easy to compensate for being too cold by adding layers of clothing, it is less easy to cool oneself as customs limit the amount of clothing that can be acceptably removed in public. It is additionally problematic that the removal of clothing increases a person's exposure to the sun, and related problems such as sunburn. Exposure to heat for prolonged periods of time can cause illness, such as heat exhaustion and heatstroke, or possibly even cause death in humans and animals. These events can occur when one's body cannot keep itself cool. The body normally has keeps itself cool by letting heat escape through the skin and by evaporating sweat.

Overheating is also experienced by animals, particularly those with little or no choice in when they exert themselves. For example, horses infrequently are given the option of deciding when their riders wish to ride them. Dogs often will patrol a property during the hottest part of the day as their instinct to protect their territory overrides their desire to remain cool. Animals, however, lack the ability to speak with their humans and thereby convey their discomfort.

As such, it is desirable to have a garment that can be worn by humans and animals alike which provides a cooling effect, and does so in a stylish, low cost, and efficient manner. The evaporative cooling garment discussed in this patent provides just such a device.

Evaporative cooling works on the premise that when water evaporates from a surface, that surface becomes cooler because heat is expended to change the water from a liquid into a vapor. As heat is used up to evaporate water from at or near the skin's surface, the over effect is a localized cooling of the skin. A nice breeze on a hot day cools humans and animals because the current of air makes perspiration evaporate quickly. The effectiveness of evaporative cooling products, and of this invention is particular, is based on its ability to absorb heat from the body and release that heat to the outside environment. The water that is absorbed by the batting material provides a heat transfer medium for this effect. As the heat accumulates in the material, the heat causes water to evaporate from the products. The direction of this heat is to the outside because the moisture differential is less to the outside (the environment next to the body is generally at a high relative humidity). On hot, humid days however, the evaporation of sweat is slowed by the increased moisture in the air. When sweating does not provide adequate “heat loss” to cool one's body through evaporative cooling, the body temperature rises. If the body does not cool properly or does not cool enough, the chance of one suffering a heat-related illness increases. Many people are susceptible to heat related illnesses, especially the very young and very old.

Evaporative cooling is an initial treatment for patients with heat exhaustion as it involves stabilization to avoid progress to heat stroke. Immediate initiation of rapid and effective cooling is crucial to treating a patient with heatstroke. Efficient and rapid reduction of the body's core temperature is a vital factor in the successful or unsuccessful outcome of treatment for heatstroke. Studies have found a trend towards improved survival in patients cooled to a core temperature below 38.9 C within 60 minutes. Other reports have shown improved survival when patients where cooled to the same level within 30 minutes. These studies indicate that heat related illness can be minimized when a person's core temperature is reduced as quickly as possible.

Symptoms of certain non-heat related illnesses can also be minimized by reducing body temperature. Multiple sclerosis (MS) is a very debilitating disease for those who suffer its affliction. Manifestation of its symptoms often varies with environmental conditions, seasons of the year, and the patient's activities. A very common complaint from MS patients is the decrease in strength and onset of fatigue concurrent with increases in climatic temperature and/or core temperature. Decreasing the core temperature of these patients has been shown to reduce the effects of elevated temperature. Additionally, sustained cooling has been shown to reduce skin temperature and reduce overall muscle spindle activity. This is also important to MS patients as this cooling could enable patients to function and perform activities more efficiently.

Humans are not the only ones susceptible to heat related symptoms and illnesses, as animals are also at risk. In fact, animals are often at greater risk to suffer heat related illnesses because many animals often rely on humans for care, and cannot communicate any heat-related distress as eloquently as humans. If the human care-taker is not fully cognizant of the animal's symptoms or conditions in heated temperatures, the animals may not receive the proper cooling treatment to prevent heat related illnesses.

This is particularly true of domesticated animals used for physically demanding labor. A prime example is the horse. Racehorses are routinely trained and raced at specific times during the summer months. At the Del Mar Racetrack in Del Mar, for example, on any given day a hundred or so horses may participate in races, the starting times of which are set months in advance. These races will occur regardless of the ambient temperature and relative humidity at the time of the race; thus it is in the horse owner's best interests to have cooling garments ready for the racehorse before and after the race. For horses used for recreational purposes, they too have little control over when their rider wants to take them for a ride. A commonly overlooked danger to horses is Anhidrosis, which is a condition in which the horse cannot sweat. A horse suffering from Anhidrosis is in obvious danger of risking heat-related injuries or even death when it exerts itself under hot or even warm conditions.

Another example of a domesticated animal that sometimes suffers from heat stress is the dog. Because different varieties of dogs evolved under different climate regimes and different breeding pressures, today's dogs come in a variety of sizes and with a variety of hair lengths. As a smaller body will tend to lose heat at a relatively greater rate than a larger body due to the physics of body surface areas and volumes, larger dogs with long coats are particularly vulnerable to heat-related injuries and illnesses. Show dogs are particularly vulnerable, as often the buildings in which dogs shows are held have poor or little air conditioning.

Thus, it is also desirable to have an evaporative cooling garment that can be easy applied to an animal, which is easy to use, will provide the animal with a substantial time period during which the animal will be cooled by the garment, and which the animal will not reject as being too uncomfortable.

Although several garment options are available for cooling humans and animals, these options have limitations. For example, humans often wet towels or other cloths to wrap around various body parts to combat heat. While this method may achieve the desired effect, towels do not provide for flexibility in movement and are susceptible to drying at a rapid rate. Also, towels do not provide a convenient garment that can be secured to a body that allows for flexible movement without the towel falling off. Another attempt to keep humans cool has been the creation of lightweight and breathable clothing. However, this clothing only helps to prevent heat buildup rather than actually cooling the wearer.

Various garments also exist for animals, such as the horse blankets taught by German Patent Application Nos. DE 20 2005 012 350 U1 2005.11.24 and DE 20 2005 012 352 U1 2005.11.24 to Busse Sportartikel. These inventions, however, do not provide for cooling of the horse during times of hot weather or after periods of exercise.

The concept of evaporative cooling garments is not unique. One category of such garments is the complex garments utilizing channels or chambers to direct the cooling efforts. For example, US Patent Application No. 2003/0208831 to Lazar, et. al., discloses an evaporative cooling garment manufactured from a multi-layered fabric with chambers where holes created by sewing or stitching channel the water into the chambers. Other evaporative cooling inventions are taught by U.S. Pat. Nos. 6,295,648, 6,276,155 and 6,257,011 to Siman-Tov, et. al. These patents provide a shirt with channels which absorb sweat and/or evaporative liquid and a device to then move air through the channels to create the cooling effect. While these inventions may provide evaporative cooling to a human or animal user of the invention, they lack the simplicity (and, inherent to the simplicity, the low cost and ease of use) of the current invention. Another evaporative cooling garment is found in US Patent Application No. 2005/0246826 to McCarter, et. al. This invention requires an exhaust fan to blow pressurized air through a plurality of “ridge and channel ribs”. Another complex evaporative cooling device is illustrated in U.S. Pat. No. 5,438,707 to Horn, which discusses a series of hollow tubes networking within a vest which convey pressurized air and create evaporative cooling. A similar idea is taught by US Patent Application No. 2004/128747 to Bumbarger, et. al., in which a reservoir of water can be used to drink or dissipate the water for evaporative cooling purposes. Another patent illustrating the complexity of inventions which require a coolant supply is U.S. Pat. No. 5,715,533 to Stein. This patent teaches a baseball cap with an interconnected series of veins through which coolant from a reservoir attached to the side of the baseball cap can flow to cause evaporative cooling.

While these garments do provide evaporative cooling to the wearers of the garments, the complexity of these inventions causes them to be considerably more expensive and difficult to use than the current invention. For example, any evaporative cooling device requiring a reservoir or power supply to activate a fan requires a user of those inventions to keep the fluids up and batteries charged. Additionally, any garment requiring tubes, channels or other means of directing air or water requires a substantially greater amount of design costs, will take more expensive materials, and will eventually cost more than the invention proposed herein.

Another evaporative cooling garment is described in U.S. Pat. No. 6,473,910 to Creagan, et. al. This invention calls for three layers of fabric, each with specific characteristics, where the layers are bonded together by heating various regions sufficiently for the layer to melt and resolidify to form inter-fiber bonds. While this garment provides evaporative cooling in what appears to be a fairly economical manner, it requires the heating and melting of the various layers to join them together, as opposed to the current invention which merely relies upon stitching to maintain each layer is a position proximate to the next. A similar invention is found in US Patent Application No. 2002/0069448 to Appolonia, which provides an evaporative material comprised of three layers of fabric. This invention, however, calls for two of the layers to have more than 1 component, which is more complex that the current invention and, therefore is not as simple to manufacture and it would be more difficult and expensive to manufacture.

Another category of evaporative cooling garments are basically cooling undershirts, designed to be worn under body armor or other similarly bulky and non-breathable devices. For example, US Patent Application No. 2007/000001 provides an undergarment which allows for passive air circulation—and thereby allows for evaporative cooling—based upon a three-dimensional honeycomb structure which is strong enough to maintain the open air passages even when compressed under body armor. U.S. Pat. No. 4,170,793 to O'Brien also teaches an evaporative cooling undergarment for use under body armor which is equally complex and reliant upon strong, resilient materials.

While the structural rigidity of these inventions may be necessary to maintaining air passages under the weigh of body armor, such advanced designs (and highly priced products) are not necessary for the human and animal evaporative cooling market the current invention services.

When dealing with animals, a primary cooling method is to provide a shady area for the animal to rest, such as a under a tree or within a stable or a barn. In many cases however, shady areas do not exist or are expensive to build. Further, shady areas limit the animal's ability to travel to a location that is not protected from direct sunlight, exposure to which can cause heat related illness. Therefore, providing a cooling garment for animals, such as a dog, horse, cow or any other domesticated animal or animal in a zoo, is preferable because of the improved cooling capabilities and flexibility in movement it provides. However, many cooling garments are not designed to properly fit animals or accommodate the amount of movement generated by most animals.

The prior art has examples of evaporative cooling garments made especially for animals, but these are expensive and considerably more complex than the current invention.

In the field of evaporative cooling devices for horses, there is at least one device on the market, known as the Recoolx horse-boots. This device appears to use evaporative cooling through the use of a series of chambers which allow the horse's leg to flex without unduly bending the horse-boot. The device, however, is quite complex in its construction and only offers a decrease of 3-5 degrees centigrade to the treated area, as opposed to the 20-30 degree drop experienced by users of the current invention.

Recoolx' product is also considerably more complex and costly than is Gordon's invention. Recoolx' horse-boots also advertise a necessary soaking time of 20 minutes, while the current invention becomes fully saturated in 20-30 seconds, and can be “refreshed” with additional soakings of 20 seconds or less.

There are also a number of apparently unpatented evaporative cooling garments which have recently appeared for sale on the internet, including Eastnova Electronic Company Ltd.'s Cooling Vest, Heliohealth's Evaporative Body Cooling Garment, and Polar's Evaporative Cooling Garments. All of these products, however, require, as stated in their instructions, between one to three minutes of immersion in water before they are functional. The current invention, on the other hand, requires only 20-30 seconds of immersion in water due to its use of superabsorbent materials.

Therefore, there is a current need for a convenient to use, cost-effective and flexible cooling garment that can help cool body temperature for a prolonged period and can safely, securely, and comfortably attach to various different body regions of both humans and animals alike.

This invention allows humans in a wide range of professions to cool themselves off over a long period of time with minimal investment of effort. Such users could include military personnel in hot environments or cramped quarters, athletes or casual sport participants, medical personnel and patients, contractors, law enforcement, border patrol, park rangers and other people who work outside of buildings, and other humans who either work in hot weather environments or work inside of an enclosed space where temperatures get uncomfortably hot.

In this respect, before explaining at least one embodiment of the invention in detail it is to be understood that the embodiments of the invention are not limited in their application to the details of construction and to the arrangement of the components set forth in the following description or illustrated in the drawings. The embodiments of the invention are capable of being practiced and carried out in various ways. In addition, it is to be understood that the phraseology and terminology employed herein are for the purpose of description and should not be regarded as limiting.

### BRIEF SUMMARY OF INVENTION

The preferred embodiment of the invention provides a wearable garment that can be used to cool humans and animals. The garment contains a body portion and an attachment portion. The body portion is generally defined by at least one outer seam. The body portion contains three layers—an outer layer, a middle layer, and an inner layer. The outer layer is comprised of a breathable fabric. The middle layer is comprised of a substantially hydrophilic material. The inner layer is substantially hydrophobic, preferably comprising a layer of finely woven substantially hydrophobic fibrous material. The attachment portion contains at least one means for attaching the garment to a wearer. The garment can be shaped to fit various shaped and sized regions of both humans and animals, including heads, legs, tails, torso regions, necks, and arms.

To activate the cooling process, the garment is first immersed in water for 20 to 30 seconds wherein the inner material absorbs the water. The wearer then wrings the excess water from the garment and places the garment on the desired body region to be cooled. Tests have shown that the cooling effect achieved by the garment can last between 2-3 hours at 114 degrees Fahrenheit. If the garment is used indoors and not placed in direct sunlight, the cooling effect can last up to seven hours. The garment can be recharged to continue the cooling process by again immersing the garment in water, for approximately one minute, then wringing the excess water from the garment and placing it on the desired area to be cooled.

Another embodiment of the invention provides a garment that can be used to cool humans and animals. The garment contains a body portion. The body portion is generally defined by at least one outer seam. The body portion contains three layers—an outer layer, a middle layer, and an inner layer. The outer layer is comprised of a breathable fabric. The middle layer is comprised of a substantially hydrophilic material. The inner layer is substantially hydrophobic, preferably comprising a layer of finely woven substantially hydrophobic fibrous material. The garment is primarily configured to serve as a cooling mat or blanket for humans and animals.

To activate the cooling process, the garment is first immersed in water for approximately 20 to 30 seconds, wherein the inner material absorbs the water. The wearer then wrings the excess water from the garment and places the garment on the desired body region to be cooled. Tests have shown that the cooling effect achieved by the garment can last between 2-3 hours at 114 degrees Fahrenheit. If the garment is used indoors and not placed in direct sunlight, the cooling effect can last up to seven hours. The garment can be recharged to continue the cooling process by again immersing the garment in water, for approximately one minute, then wringing the excess water from the garment and placing it on the desired area to be cooled.

Another embodiment of the invention provides a wearable garment that can be used to cool humans and animals. The garment contains a body portion and at least one means for attaching the body portion around a human or animal. The body portion is generally defined by at least one outer seam. The body portion contains three layers—an outer layer, a middle layer, and an inner layer. The outer layer is comprised of a breathable fabric. The middle layer is comprised of a substantially hydrophilic material. The inner layer is substantially hydrophobic, preferably comprising a layer of finely woven substantially hydrophobic fibrous material. The at least one means for attaching the body portion around a human or animal can comprise a snap, button, Velcro, hook and loop, or other means for attachment. The garment can be shaped to fit various shaped and sized regions of both humans and animals, including heads, legs, torso regions, necks, and arms.

To activate the cooling process, the garment is first immersed in water for approximately 20 to 30 seconds, wherein the inner material absorbs the water. The wearer then wrings the excess water from the garment and places the garment on the desired body region to be cooled. Tests have shown that the cooling effect achieved by the garment can last between 2-3 hours at 114 degrees Fahrenheit. If the garment is used indoors and not placed in direct sunlight, the cooling effect can last up to seven hours. The garment can be recharged to continue the cooling process by again immersing the garment in water, for approximately one minute, then wringing the excess water from the garment and placing it on the desired area to be cooled.

The three layers of which the garment is made are kept in close proximity to one another through quilted stitching. Rather than relying upon the more expensive and time consuming methods of maintaining the integrity of the various layers through melting and other means, the current invention merely uses stitching in a quilt-like pattern to secure the layers to each other and to ensure that parts do not sag to the bottom of a compartment and decrease the efficiency of the garment. It should also be noted that by quilting the layers together, a manufacturer of the invention has the option of using different stitching patterns, or even putting a logo or advertising insignia on the garment.

The short time period (relative to the prior art) which is needed to activate the invention is due to the superabsorbency of the middle layer. A preferred embodiment of the invention calls for this layer to be comprised of material with a horizontal wicking rate of 60 mm, and a saturation rate of 1700% of the weight of the absorbing material within 10 seconds of contact with water. A particularly preferred material is DT275 from Concert Industries Ltd., of Gatineau, Canada, which has a base weight of 275 gsm, thickness of 3.4 mm, tensile MD of 1500 g/50 mm, density of 0.09 g/cm cubed,

There has thus been outlined, rather broadly, features of the embodiments of the invention in order that the detailed description thereof may be better understood, and in order that the present contribution to the art may be better appreciated. There are additional features of the embodiments of the invention that will be described hereinafter and which will form the subject matter of the claims appended hereto.

### BRIEF DESCRIPTION OF THE DRAWINGS

The accompanying drawings, which are incorporated in and form a part of this specification, illustrate embodiments of the invention and together with the description, serve to explain the principals of the embodiments of the invention.

FIG. 1 shows a top view of one embodiment of the flexible cooling garment for attachment to the body of an animal.

FIG. 1A shows the cross-section view, along the line A-A′ in FIG. 1, of the body portion of one embodiment of the flexible cooling garment for attachment to the body of an animal.

FIG. 2 shows a side view of one embodiment of the flexible cooling garment for attachment to the body of an animal.

FIG. 3 shows a top view of another embodiment of the flexible cooling garment for attachment to the head of an animal.

FIG. 4 shows a top view of another embodiment of the flexible cooling garment for attachment to various body regions.

FIG. 5 shows a top view of another embodiment of the flexible cooling garment for use as a blanket.

FIG. 6 shows a top view of another embodiment of the flexible cooling garment for attachment to the body region of a human.

FIG. 7 shows a top view of another embodiment of the flexible cooling garment for placement around the neck of a human or animal.

FIG. 8 shows a top view of another embodiment of the flexible cooling garment for attachment around the leg of a human or animal.

### DETAILED DESCRIPTION OF THE INVENTION

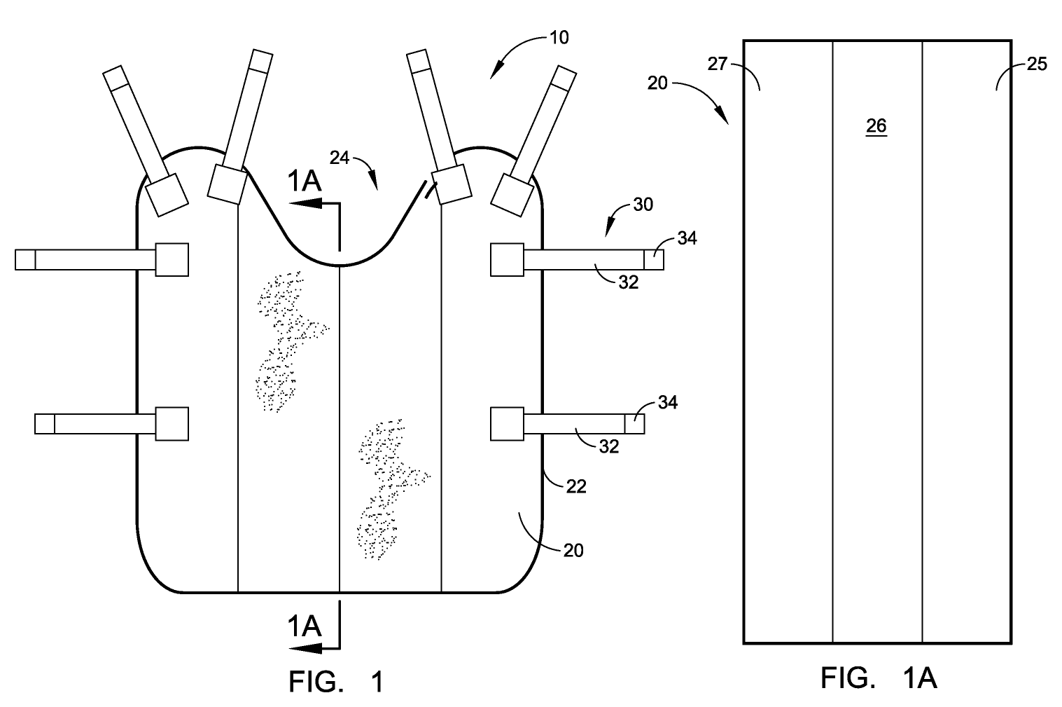

Referring now to the drawings, wherein similar parts are identified by like reference numerals, FIG. 1 shows a top view of an embodiment of the flexible cooling garment 10 for attachment to the body of an animal (not shown). Garment 10 includes a body 20 and at least one attachment portion 30. Body 20 is preferably lightweight and flexible. Body 20 is generally defined by an outer seam 22. Outer seam 22 can define body 20 in various shapes and sizes to accommodate various animals. As shown, outer seam 22 defines a primarily rectangular shaped body 20 with a head opening 24, defining a garment 10 that is used to cover an animal such as a dog or horse.

Attachment portion 30 preferably includes a strap 32 and a connector portion 34. Strap 32 can be comprised of any type of flexible fabric and can be adjustable to fit various sized animals. Connector portion 34 is designed to engage the connector portion 34 located on the end opposite end of the garment 10. Connector portion 34 can be a variety of different connectors including, but not limited to hooks, buttons, snaps, and Velcro. Garment 10 can include more than one attachment portion 30 to help secure garment 10 on the body of an animal.

FIG. 1A shows the cross-section view, along the line A-A′ in FIG. 1, of body 20. This figure illustrates the nature of material used for body 20. Body 20 is comprised of an inner layer 25, a middle layer 26, and an outer layer 27. Inner layer 25 is preferably comprised of a material having substantially hydrophobic characteristics. This material can be either comprised of a substantially hydrophobic material or be a material that is closely woven so as to give body 20 substantially hydrophobic characteristics. For example, inner layer 25 can comprise closely woven nylon fibers. Middle layer 26 is preferably comprised of a substantially hydrophilic material as would be recognized by one with ordinary skill in the art, including but not limited to fine acrylic fibers, terry cloth, cotton, or other substantially hydrophilic materials. Outer layer 27 is preferably comprised of a breathable material as would be recognized by one with ordinary skill in the art, such as nylon. Inner layer 25, middle layer 26, and outer layer 27 can be joined together to form body 20 in various different manners.

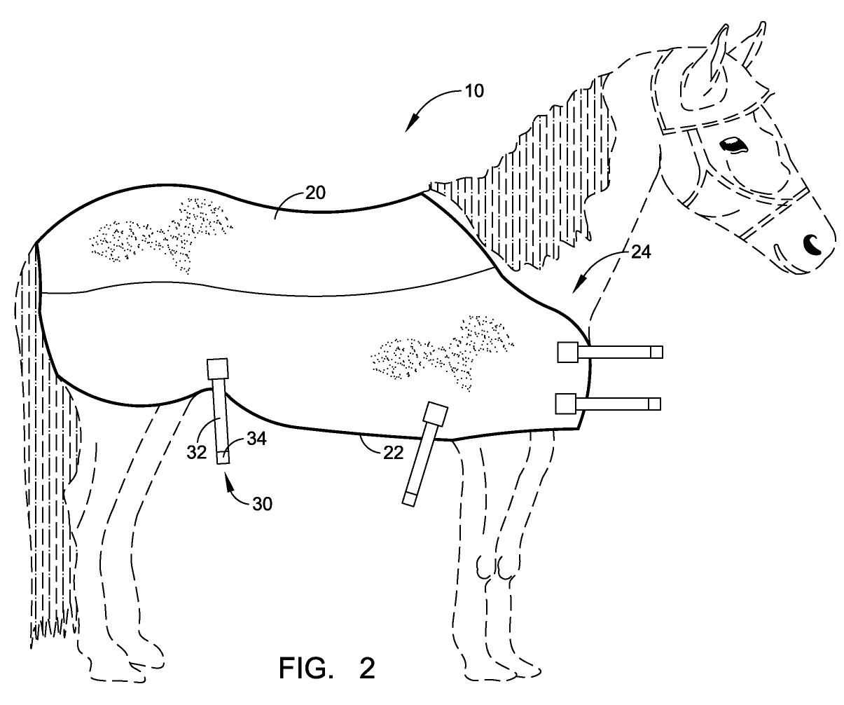

FIG. 2 shows a side view of an embodiment of the flexible cooling garment 10 for attachment to the body of an animal (not shown), including body 20, outer seam 22, head opening 24, attachment portion 30, strap 32, and connector portion 34.

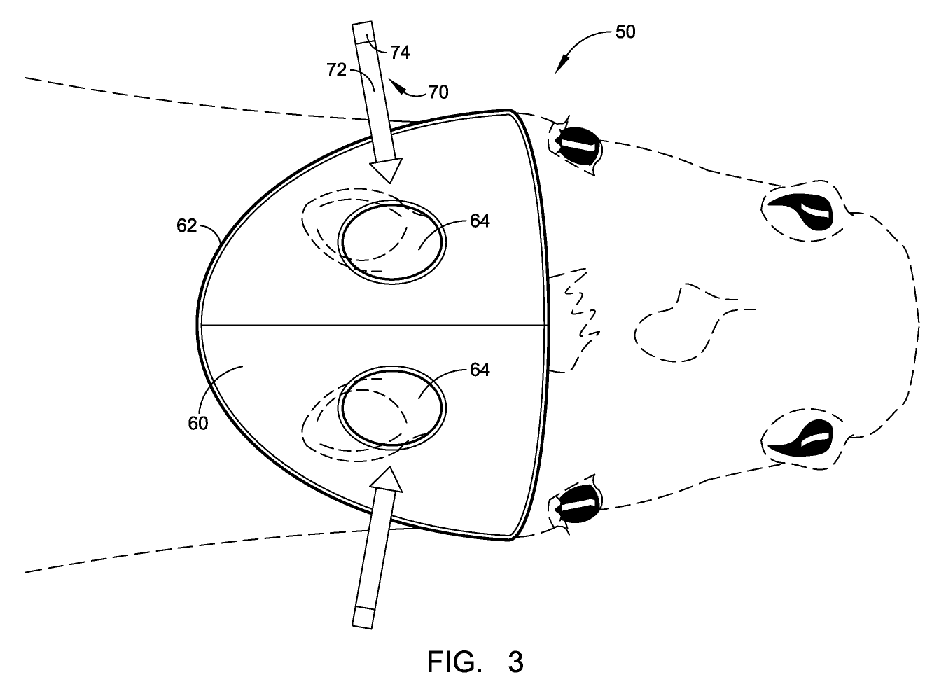

FIG. 3 shows a top view of another embodiment of a flexible cooling garment 50 for attachment to the head of an animal (not shown). Garment 50 includes a body 60 and at least one attachment portion 70. Body 60 is preferably lightweight and flexible. Body 60 is preferably comprised of an inner layer, a middle layer, and an outer layer, as shown and described in FIG. 1A and the related description thereof. Body 60 is generally defined by an outer seam 62. Outer seam 62 can define body 60 in various shapes and sizes to accommodate various animals. As shown, outer seam 62 defines a primarily oval shaped body 60 with two eye openings 64, defining a garment 50 that is used to partially cover the head of an animal such as a dog or horse.

Attachment portion 70 preferably includes a strap 72 and a connector portion 74. Strap 72 can be comprised of any type of flexible fabric and can be adjustable to fit various sized animals. Connector portion 74 is designed to engage the connector portion 74 located on the end opposite end of the garment 10. Connector portion 74 can be a variety of different connectors including, but not limited to hooks, buttons, snaps, and Velcro. Garment 50 can include more than one attachment portion 70 to help secure garment 50 on the head of an animal.

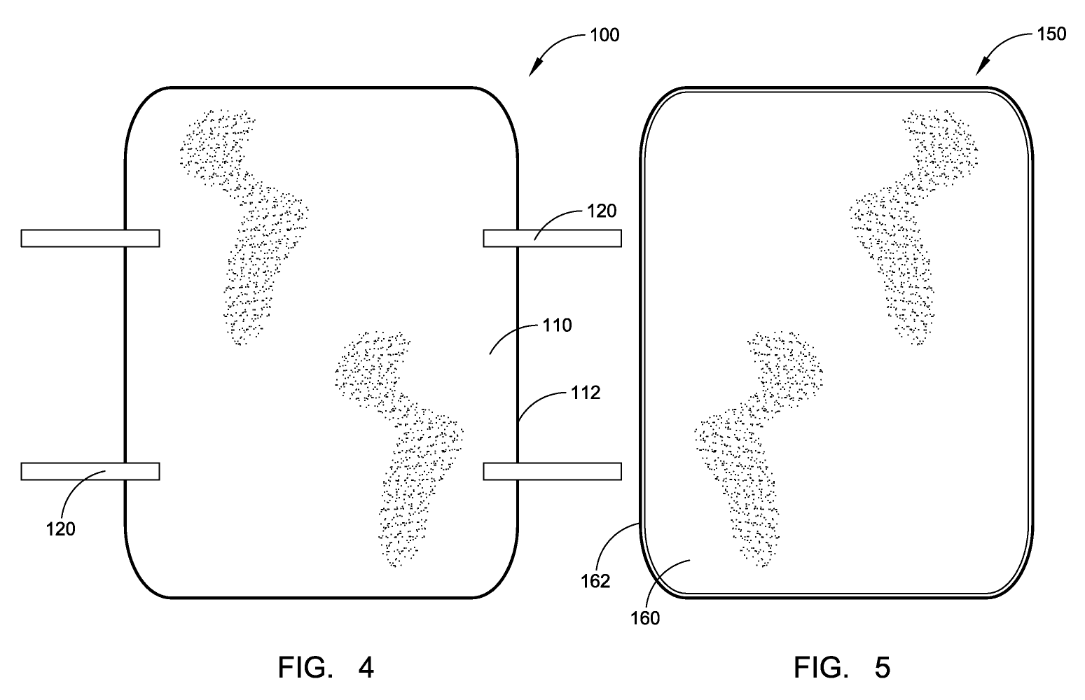

FIG. 4 shows a top view of another embodiment of a flexible cooling garment 100 for attachment to various body regions of a human or animal (not shown). Garment 100 includes a body 110 and at least one attachment portion 120. Body 110 is preferably lightweight and flexible. Body 110 is preferably comprised of an inner layer, a middle layer, and an outer layer, as shown and described in FIG. 1A and the related description thereof. Body 110 is generally defined by an outer seam 112. Outer seam 112 can define body 110 in various shapes and sizes to accommodate various animals. As shown, outer seam 112 defines a primarily rectangular shaped body 110, defining a garment 110 that is used to partially cover various regions of a human or animal.

Attachment portion 120 can be comprised of any type of flexible fabric and can be adjustable to fit various sized humans or animals. Attachment portion 120 can be comprised of Velcro material, so as to engage the connector portion 124 located on the end opposite end of the garment 100. There can be more than one attachment portion 120 to help secure garment 100 on a human or animal.

FIG. 5 shows a top view of another embodiment of the flexible cooling garment 150. Garment 150 can be used for a variety of applications, including use as a cooling blanket for either humans or animals. Garment 150 includes a body 160. Body 160 is preferably lightweight and flexible. Body 160 is preferably comprised of an inner layer, a middle layer, and an outer layer, as shown and described in FIG. 1A and the related description thereof. Body 160 is generally defined by an outer seam 162. Outer seam 162 can define body 160 in various shapes and sizes to accommodate various humans and animals. As shown, outer seam 162 defines a primarily rectangular shaped body 160, defining a garment 150 that can be used as a cooling mat or blanket.

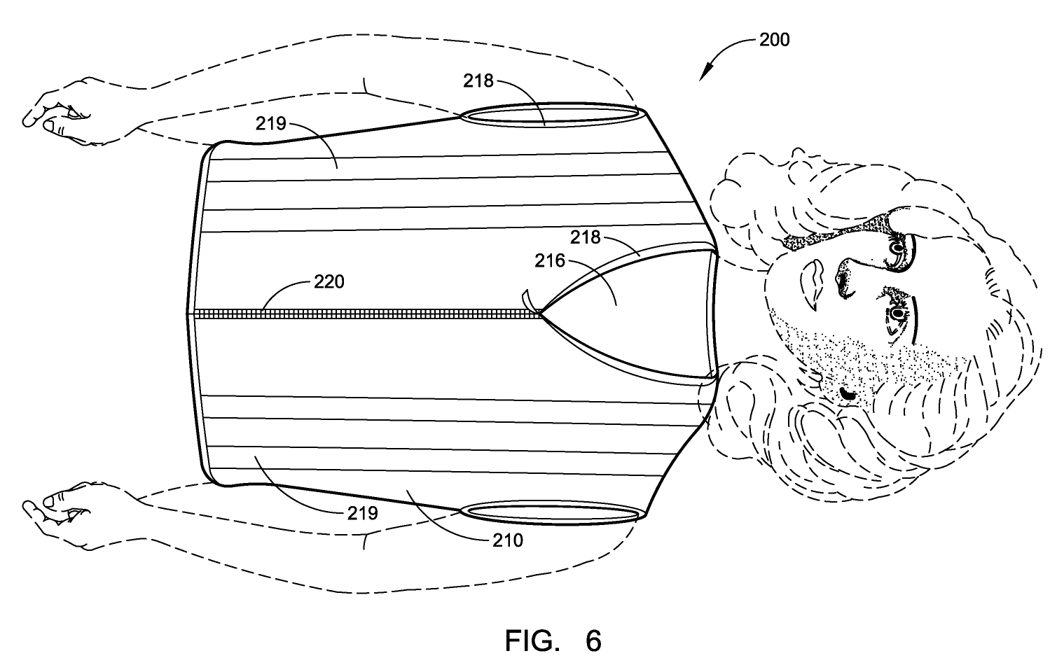

FIG. 6 shows a top view of another embodiment of a flexible cooling garment 200 for attachment to the body region of a human (not shown). Garment 200 includes a body 210 and at least one attachment portion 220. Body 210 is preferably lightweight and flexible. Body 210 is preferably comprised of an inner layer, a middle layer, and an outer layer, as shown and described in FIG. 1A and the related description thereof. Body 210 is generally defined by an outer seam 212. Outer seam 212 can define body 210 in various shapes and sizes to accommodate various animals. As shown, outer seam 212 defines a body 210 with a head opening 216 and arm openings 218, defining a garment 200 that is used to partially cover the body region of a human. Although garment 200 is shown as a vest-type garment, garment 200 can include sleeves and/or a collar to resemble a shirt, long-sleeved shirt, vest, or other garment that can be worn around the body of a human as would be recognized by one with ordinary skill in the art.

Further, body 210 can include one or more reflective surfaces 219 to help identify a wearer of garment 200 at night. Body 210 also includes an attachment portion 220 that is used to help secure garment 200 on the body of a wearer. As shown, attachment portion 220 is comprised of a Velcro strip running upward along the middle of the front of body 210 from outer seam 212 to head opening 216. However, attachment portion 220 can also be comprised of one or more straps having connector portions attached thereto, or buttons, hooks and loops, snaps, or other attachment means as would be recognized by one with ordinary skill in the art, to help secure garment 200 to the body of a wearer.

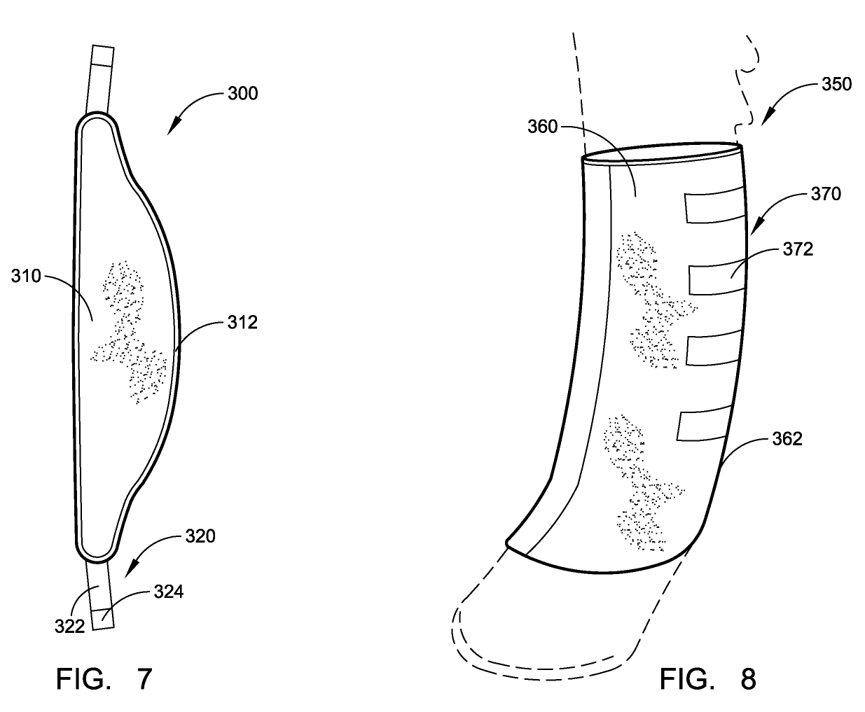

FIG. 7 shows a top view of another embodiment of a flexible cooling garment 300 for placement around the neck of a human or animal (not shown). Garment 300 includes a body 310 and at least one attachment portion 320. Body 310 is preferably lightweight and flexible. Body 310 is preferably comprised of an inner layer, a middle layer, and an outer layer, as shown and described in FIG. 1A and the related description thereof. Body 310 is generally defined by an outer seam 312. Outer seam 312 can define body 310 in various shapes and sizes to accommodate various animals. As shown, outer seam 312 defines a primarily rectangular shaped body 310, defining a garment 310 that is used for placement around the neck of a human or animal.

Attachment portion 320 can be comprised of any type of flexible fabric and can be adjustable to fit various sized humans or animals. Attachment portion 320 can be comprised of Velcro material, so as to engage the connector portion 324 located on the end opposite end of the garment 300. There can be more than one attachment portion 320 to help secure garment 300 on a human or animal.

FIG. 8 shows a top view of another embodiment of a flexible cooling garment 350 for attachment around the leg of a human or animal (not shown). Garment 350 includes a body 360 and at least one attachment portion 370. Body 360 is preferably lightweight and flexible. Body 360 is preferably comprised of an inner layer, a middle layer, and an outer layer, as shown and described in FIG. 1A and the related description thereof. Body 360 is generally defined by an outer seam 362. Outer seam 362 can define body 360 in various shapes and sizes to accommodate various animals. As shown, outer seam 362 defines a primarily rectangular shaped body 360, defining a garment 350 that is used for attachment around the leg of a human or animal. Body 360 can also define an opening (not shown) for positioning around the knee of a human or animal to allow bending of the leg of the human or animal.

Attachment portion 370 can be comprised of any type of flexible fabric and can be adjustable to fit various sized humans or animals. Attachment portion 370 can be comprised of Velcro material, so as to engage the connector portion 374 located on the end opposite end of the garment 350. There can be more than one attachment portion 370 to help secure garment 350 on a human or animal.

With respect to the above description it is to be realized that the optimum dimensional relationships for the parts of the invention, including variations in size, materials, shape, form, function and manner of operation, assembly, and use, are deemed readily apparent and obvious to one skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents fall within the scope of the present invention.

The above description, together with the advantages of the invention and the various features of novelty which characterize the invention, are pointed out with particularity in the claims annexed to and forming a part of this disclosure. For a better understanding of the invention, its operating advantages and the specific advantages attained by its uses, reference should be made to the accompanying drawings and descriptive matter in which there are illustrated preferred embodiments of the invention.

Further, the purpose of the foregoing abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting, as to the scope of the invention in any way.

What is claimed is:

1. A flexible garment comprising: a body portion, the body portion defined by an outer seam, the body portion including a substantially hydrophobic inner layer, a substantially hydrophilic middle layer, and an outer layer; means of maintaining the inner layer, middle layer, and outer layer in close proximity to each other; and at least one attachment portion whereby the attachment portion allows a user of the invention to attach the body portion to a living human or animal; whereby the garment can be placed in a liquid for a certain period, wherein the middle layer retains at least some of the liquid, and the garment is removed from the liquid and can be used to cool at least one body portion of a human or animal.

2. The flexible garment of claim 1, where the substantially hydrophilic middle layer can be saturated in water by dipping the flexible garment in water for 30 seconds, to the point where the garment weighs at least 15 times its original weight after soaking in water for 30 seconds.

3. The flexible garment of claim 2, where the substantially hydrophilic middle layer has an absorbance rate such that after a time period has elapsed after the garment has last been submerged in water, where the time period is between one and three hours, the garment can be resubmerged in water for 20 seconds, and weigh at least 15 times its original weight after soaking in water for 20 seconds.

4. The flexible garment of claim 3, where the means of maintaining the inner layer, middle layer, and outer layer in close proximity to each other consists of stitching.

5. The flexible garment of claim 3, where the substantially hydrophobic inner layer, the substantially hydrophilic middle layer, and the outer layer each consist of one layer of material.

6. The flexible garment of claim 5, where the substantially hydrophilic middle layer has a horizontal wicking rate of 60 mm or more and a saturation rate of 1700% of the weight of the absorbing material within 10 seconds of contact with water.

7. The flexible garment of claim 5, where the substantially hydrophilic middle layer has a base weight of 275 gsm, thickness of 3.4 mm, tensile MD of 1500 g/50 mm, and a density of 0.09 g/cm cubed.

8. The flexible garment of claim 5, where the substantially hydrophilic middle layer has a horizontal wicking rate of 60 mm or more and a saturation rate of 1700% of the weight of the absorbing material within 10 seconds of contact with water, and where the substantially hydrophilic middle layer has a base weight of 275 gsm, thickness of 3.4 mm, tensile MD of 1500 g/50 mm, and a density of 0.09 g/cm cubed.

9. The flexible garment of claim 1, where a temperature decrease of at least 10 degrees Centigrade can be achieved on a surface region of a desired body part by soaking the flexible garment in water for 30 seconds and placing the body portion of the flexible garment on the desired body part for less than 5 minutes.

10. The flexible garment of claim 1, where a temperature decrease of at least 20 degrees Centigrade can be achieved on a surface region of a desired body part by soaking the flexible garment in water for 30 seconds and placing the body portion of the flexible garment on the desired body part for at less than 5 minutes.

11. The flexible garment of claim 1, where a temperature decrease of at least 20 degrees Centigrade can be achieved on a surface region of a desired body part by soaking the flexible garment in water for 30 seconds and placing the body portion of the flexible garment on the desired body part for at less than 2 minutes.

12. The flexible garment of claim 1, where substantially hydrophilic middle layer has a horizontal wicking rate of 60 mm and a saturation rate of at least 15 times the weight of the absorbing material within 10 seconds of contact with water.

13. The flexible garment of claim 1, where the substantially hydrophilic middle layer has a base weight of 275 gsm, thickness of 3.4 mm, tensile MD of 1500 g/50 mm, density of 0.09 g/cm cubed,

14. The flexible garment of claim 1, where the substantially hydrophilic middle layer can be saturated in water by dipping the flexible garment in water for one minute or more, and where the means of maintaining the inner layer, middle layer, and outer layer in close proximity to each other consists of stitching, and where the substantially hydrophobic inner layer consists of one layer of material, and where a temperature decrease of at least 10 degrees Centigrade on a surface region of a desired body part can be achieved by soaking the flexible garment in water for less than 45 seconds and placing the body portion of the flexible garment on the desired body part for less than 5 minutes.

15. The flexible garment of claim 1, where the substantially hydrophilic middle layer can be saturated in water by dipping the flexible garment in water for one minute or more, and where the means of maintaining the inner layer, middle layer, and outer layer in close proximity to each other consists of stitching, and where the substantially hydrophobic inner layer consists of one layer of material, and where a temperature decrease of at least 20 degrees Centigrade on a surface region of a desired body part can be achieved by soaking the flexible garment in water for less than 45 seconds and placing the body portion of the flexible garment on the desired body part for less than 5 minutes.

16. A flexible garment consisting of: a body portion, the body portion defined by an outer seam, the body portion including a substantially hydrophobic inner layer, a substantially hydrophilic middle layer, and an outer layer; means of maintaining the inner layer, middle layer, and outer layer in close proximity to each other; and at least one attachment portion whereby the attachment portion allows a user of the invention to attach the body portion to a living human or animal; whereby the garment can be placed in a liquid for a certain period, wherein the middle layer retains at least some of the liquid, and the garment is removed from the liquid and can be used to cool a user, where the substantially hydrophilic middle layer can be saturated in water by dipping the flexible garment in water for 30 seconds, to the point where the garment weighs at least 15 times its original weight after soaking in water for 30 seconds, and, where the substantially hydrophilic middle layer has an absorbance rate such that after a time period has elapsed after the garment has last been submerged in water, where the time period is between one and three hours, the garment can be resubmerged in water for 20 seconds and weigh at least 15 times its original weight after soaking in water for 20 seconds.

17. A method of cooling a desired body part, comprising the steps of, first, obtaining a flexible garment consisting of: a body portion, the body portion defined by an outer seam, the body portion including a substantially hydrophobic inner layer, a substantially hydrophilic middle layer, and an outer layer; means of maintaining the inner layer, middle layer, and outer layer in close proximity to each other; and at least one attachment portion whereby the attachment portion allows a user of the invention to attach the body portion to a living human or animal; whereby the garment can be placed in a liquid for a certain period, wherein the middle layer retains at least some of the liquid, and the garment is removed from the liquid and can be used to cool a user, where the substantially hydrophilic middle layer can be saturated in water by dipping the flexible garment in water for 30 seconds, to the point where the garment weighs at least 15 times its original weight after soaking in water for 30 seconds, and, where the substantially hydrophilic middle layer has an absorbance rate such that after a time period has elapsed after the garment has last been submerged in water, where the time period is between one and three hours, the garment can be resubmerged in water for 20 seconds or less and weigh at least 15 times its original weight after soaking in water for 20 seconds, second, wringing the excess water out of the flexible garment, third, applying the flexible garment to a desired body part.

18. The method of claim 17, where the desired body part is a body part of a horse.

19. The method of claim 17, where the desired body part is a body part of a dog.

20. The method of claim 17, where the desired body part is a body part of a human.
